(19) United States
(12) Patent Application Publication
(10) Pub. No.: US 2020/0341262 A1
(43) Pub. Date: Oct. 29, 2020

(54) OBJECTIVE OPTICAL SYSTEM FOR ENDOSCOPE AND ENDOSCOPE

(71) Applicant: FUJIFILM Corporation, Tokyo (JP)

(72) Inventor: Kazuki INOUE, Saitama (JP)

(73) Assignee: FUJIFILM Corporation, Tokyo (JP)

(21) Appl. No.: 16/852,073

(22) Filed: Apr. 17, 2020

(30) Foreign Application Priority Data
Apr. 25, 2019 (JP) 2019-084142

(51) Int. Cl. G02B 23/24; G02B 13/00; G02B 9/60; G02B 27/00; A61B 1/00

(52) U.S. Cl. CPC G02B 23/243; G02B 13/0045; A61B 1/00174; G02B 27/0025; A61B 1/00096; G02B 9/60

(57) ABSTRACT

The objective optical system for an endoscope consists of, in order from an object side, a negative front group, an aperture stop, and a positive rear group. A lens closest to the object side in the front group is a negative lens concave toward an image side, and a lens positioned second from the object side in the front group is a negative lens concave toward the object side. The rear group includes a cemented lens in which a positive lens and a negative lens are cemented in order from the object side. The objective optical system for an endoscope satisfies predetermined conditional expressions relating to a focal length of a whole system, a focal length of the front group, and a distance from a lens surface closest to the object side to a lens surface closest to the image side.

### CROSS-REFERENCE TO RELATED APPLICATIONS

The present application claims priority under 35 U.S.C. § 119 to Japanese Patent Application No. 2019-084142, filed on Apr. 25, 2019. The above application is hereby expressly incorporated by reference, in its entirety, into the present application.

### BACKGROUND OF THE INVENTION

### 1. Field of the Invention

The present disclosure relates to an objective optical system for an endoscope and an endoscope.

### 2. Description of the Related Art

In the related art, various lens systems have been proposed as an objective optical system for an endoscope. WO2018/061385A discloses an endoscope objective optical system consisting of, in order from an object side, a front group having a negative refractive power, a brightness stop, and a rear group having a positive refractive power. JP5537750B discloses an endoscope objective lens comprising, in order from an object side, a front group having a negative refractive power, a brightness stop, and a rear group having a positive refractive power. JP4675348B discloses an objective optical system which is composed of, in order from an object side, a first group including two lenses with a negative refractive power and a positive refractive power, a stop, and a second group having a positive refractive power, and in which a spectral endoscope is assumed.

### SUMMARY OF THE INVENTION

It is desired that an objective optical system for an endoscope has a wide angle of view so that a wide range can be observed, and has favorable optical performance by correcting various aberrations so that a lesion portion or the like can be accurately specified. On the other hand, for reduction in patient's burden, there is a demand for an objective optical system for an endoscope to have a small size.

However, it cannot be said that the lens systems disclosed in WO2018/061385A, JP5537750B, and JP4675348B have a sufficiently short total length.

The present disclosure has been made in consideration of the above-mentioned circumstances, and an object of the present disclosure is to provide an objective optical system for an endoscope which has a wide angle of view, has a small size, and maintains favorable optical performance and an endoscope comprising the objective optical system for an endoscope.

An objective optical system for an endoscope according to an aspect of the present disclosure consists of, in order from an object side to an image side: a front group having a negative refractive power; an aperture stop; and a rear group having a positive refractive power, where a lens closest to the object side in the front group is a first lens having a negative refractive power and concave toward the image side, a lens positioned second from the object side in the front group is a second lens having a negative refractive power and concave toward the object side, the rear group includes a cemented lens in which a positive lens and a negative lens are cemented in order from the object side, and Conditional Expressions (1) and (2) are satisfied in a case where a focal length of a whole system is denoted by f, a focal length of the front group is denoted by fF, and a distance on an optical axis from a lens surface closest to the object side to a lens surface closest to the image side is denoted by L.

−2<f/fF<−1.3 (1)

3<L/f<5 (2)

In the objective optical system for an endoscope according to the above aspect, it is preferable that at least one of Conditional Expression (1-1) or (2-1) is satisfied.

−1.8<f/fF<−1.1 (1-1)

3.2<L/F<4.8 (2-1)

In the objective optical system for an endoscope according to the above aspect, in a case where a focal length of a whole system is denoted by f, and a focal length of the first lens is denoted by f1, it is preferable that Conditional Expression (3) is satisfied and it is more preferable that Conditional Expression (3-1) is satisfied.

−0.85<f/f1<−0.3 (3)

−0.8<f/f1<−0.35 (3-1)

In the objective optical system for an endoscope according to the above aspect, the cemented lens in the rear group consists of two lenses, and in a case where an Abbe number of the positive lens constituting the cemented lens at a d line is denoted by vp and an Abbe number of the negative lens constituting the cemented lens at a d line is denoted by vn, it is preferable that Conditional Expression (4) is satisfied and it is more preferable that Conditional Expression (4-1) is satisfied.

8<vp−vn<28 (4)

10<vp−vn<26 (4-1)

In the objective optical system for an endoscope according to the above aspect, in a case where a focal length of a whole system is denoted by f, and a focal length of the second lens is denoted by f2, it is preferable that Conditional Expression (5) is satisfied and it is more preferable that Conditional Expression (5-1) is satisfied.

−1.2<f/f2<−0.4 (5)

−1.1<f/f2<−0.5 (5-1)

In the objective optical system for an endoscope according to the above aspect, in a case where a focal length of a whole system is denoted by f, and a focal length of the rear group is denoted by fR, it is preferable that Conditional Expression (6) is satisfied and it is more preferable that Conditional Expression (6-1) is satisfied.

0.7<f/fR<1.5 (6)

0.8<f/fR<1.4 (6-1)

In the objective optical system for an endoscope according to the above aspect, in a case where a focal length of a whole system is denoted by f, and a focal length of the cemented lens in the rear group is denoted by fc, it is preferable that Conditional Expression (7) is satisfied and it is more preferable that Conditional Expression (7-1) is satisfied.

0.05<f/fc<0.5 (7)

0.07<f/fc<0.45 (7-1)

In the objective optical system for an endoscope according to the above aspect, it is preferable that the cemented lens in the rear group is disposed to be closest to the image side in the rear group.

In the objective optical system for an endoscope according to the above aspect, it is preferable that the second lens is a plano-concave lens or a biconcave lens.

In the objective optical system for an endoscope according to the above aspect, it is preferable that a lens closest to the object side in the rear group is a positive lens.

In the objective optical system for an endoscope according to the above aspect, the front group may include a parallel flat plate closest to the object side.

The number of lenses in the objective optical system for an endoscope according to the above aspect may be five. Further, the number of lenses in the front group may be two, and the number of lenses in the rear group may be three.

An endoscope according to another aspect of the present disclosure comprises the objective optical system for an endoscope according to the above aspect of the present disclosure.

In the present specification, the terms “consisting of ˜” and “consist of ˜” mean that the lens may include: a lens substantially having no refractive power; optical elements, which are not lenses, such as a stop, a filter, and a cover glass; a lens flange; a lens barrel; an imaging element; and the like in addition to the above-mentioned constituent elements.

In the present specification, the term “18 group having a positive refractive power” means that the group has a positive refractive power as a whole. Likewise, the term “˜ group having a negative refractive power” means that the group has a negative refractive power as a whole. The term “a lens having a positive refractive power” and the term “a positive lens” are synonymous. The term “a lens having a negative refractive power” and “a negative lens” are synonymous.

The number of lenses described above is the number of lenses that are constituent elements. For example, the number of lenses in a cemented lens composed by cementing a plurality of single lenses made of different materials is represented by the number of single lenses constituting the cemented lens. The term “single lens” means one lens that is not cemented. However, a complex aspherical lens (a lens in which a spherical lens and an aspherical film formed on the spherical lens are integrated and which functions as one aspherical lens as a whole) is treated as one lens without being regarded as a cemented lens. The sign of a refractive power and the surface shape of a lens including an aspherical surface are considered in a paraxial range unless otherwise specified.

In the present specification, the term “a whole system” means “an objective optical system for an endoscope”, and the term “a focal length” used in conditional expressions is a paraxial focal length. The values used in conditional expressions are values on a d line basis. The “d line”, “C line”, and “F line” described in the present specification are emission lines. The wavelength of the d line is 587.56 nm (nanometers), the wavelength of the C line is 656.27 nm (nanometers), and the wavelength of the F line is 486.13 nm (nanometers).

According to the present disclosure, it is possible to provide an objective optical system for an endoscope which has a wide angle of view, has a small size, and maintains favorable optical performance and an endoscope comprising the objective optical system for an endoscope.

### BRIEF DESCRIPTION OF THE DRAWINGS

FIG. 1 is a cross-sectional view corresponding to an objective optical system for an endoscope of Example 1 of the present disclosure and illustrating a configuration and rays of an objective optical system for an endoscope according to an embodiment of the present disclosure.

FIG. 2 is a cross-sectional view illustrating a configuration and rays of an objective optical system for an endoscope according to Example 2 of the present disclosure.

FIG. 3 is a cross-sectional view illustrating a configuration and rays of an objective optical system for an endoscope according to Example 3 of the present disclosure.

FIG. 4 is a diagram of aberrations of the objective optical system for an endoscope according to Example 1 of the present disclosure.

FIG. 5 is a diagram of aberrations of the objective optical system for an endoscope according to Example 2 of the present disclosure.

FIG. 6 is a diagram of aberrations of the objective optical system for an endoscope according to Example 3 of the present disclosure.

FIG. 7 is a schematic configuration diagram of an endoscope according to the embodiment of the present disclosure.

### DESCRIPTION OF THE PREFERRED EMBODIMENTS

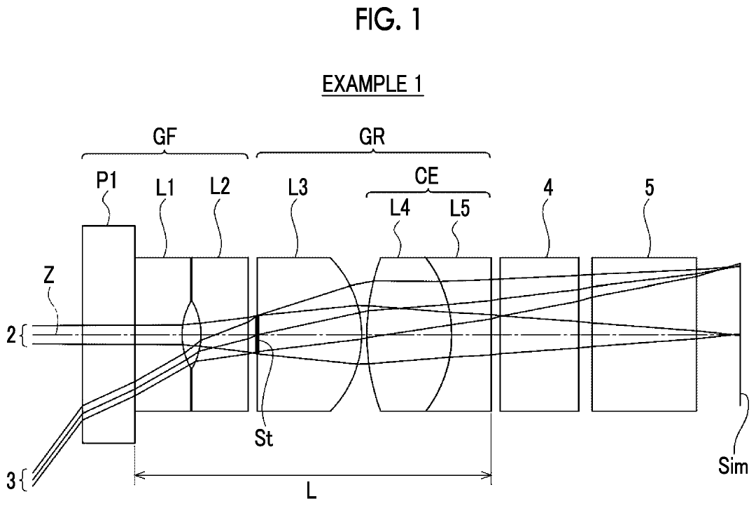

Hereinafter, embodiments of the present disclosure will be described in detail with reference to the drawings. FIG. 1 is a diagram illustrating a configuration of a cross section including an optical axis Z of an objective optical system for an endoscope according to an embodiment of the present disclosure. The example shown in FIG. 1 corresponds to Example 1 to be described later. In FIG. 1, a left side is an object side, a right side is an image side. FIG. 1 also shows rays including on-axis rays 2 and rays with the maximum angle of view 3 from an object at a finite distance.

The objective optical system for an endoscope according to the present disclosure consists of, in order from the object side to the image side along the optical axis Z, a front group GF having a negative refractive power, an aperture stop St, and a rear group GR having a positive refractive power. By disposing, in order from the object side to the image side, a lens group having a negative refractive power and a lens group having a positive refractive power, a back focal length can be ensured. As a result, there is an advantage in achieving a wide angle of view. The aperture stop St in FIG. 1 does not indicate its shape, and indicates a position of the stop on the optical axis.

In the example shown in FIG. 1, the front group GF comprises only two lenses consisting of a first lens L1 and a second lens L2 in order from the object side to the image side as lenses, and the rear group GR comprises only three lenses consisting of a third lens L3 having a positive refractive power, a fourth lens L4 having a positive refractive power, and a fifth lens L5 having a negative refractive power in order from the object side to the image side as lenses. However, the number of lenses constituting each group can be different from the number in the example shown in FIG. 1.

In the example shown in FIG. 1, the front group GF comprises a parallel flat plate P1 closest to the object side. The parallel flat plate P1 has a function as a sealing member in a case where an objective optical system for an endoscope is sealed in an endoscope. In the example shown in FIG. 1, an optical member 4 and an optical member 5 are disposed between the fifth lens L5 and an image plane Sim. The optical member 4 and the optical member 5 are members assumed to include various filters, a cover glass, and/or the like. The various filters include, for example, a low pass filter, an infrared cut filter, and a filter that cuts a specific wavelength region. The parallel flat plate P1, the optical member 4, and the optical member 5 are members having no refractive power, of which the incident surface and the exit surface are parallel, and are not lenses. A configuration in which at least one of the parallel flat plate P1, the optical member 4, or the optical member 5 is omitted is also possible.

A lens closest to the object side in the front group GF is the first lens L1 having a negative refractive power and concave toward the image side. A lens positioned second from the object side in the front group GF is a second lens L2 having a negative refractive power and concave toward the object side. Since the front group GF comprises the two negative lenses having the above-described configuration, it becomes easy to increase the angle of view while suppressing field curvature.

The first lens L1 can be a plano-concave lens of which the surface on the image side is concave, or a biconcave lens.

The second lens L2 can be a plano-concave lens of which the surface on the object side is concave, or a biconcave lens. In such a case, it is possible to contribute to suppression of field curvature.

The front group GF may be composed to comprise the parallel flat plate P1 closest to the object side. In such a case, the objective optical system for an endoscope can be sealed by the parallel flat plate P1. In a case where the objective optical system for an endoscope is sealed by the parallel flat plate P1, influence of shift and tilt caused by adhesion during sealing can be more reduced than in a case where the objective optical system for an endoscope is sealed by a lens.

It is preferable that a lens closest to the object side in the rear group GR is a positive lens. In such a case, there is an advantage in suppressing spherical aberration.

The rear group GR comprises a cemented lens CE in which a positive lens and a negative lens are cemented in order from the object side. The cemented lens CE is advantageous in suppressing lateral chromatic aberration. It is preferable that the cemented lens CE is disposed to be closest to the image side in the rear group GR. In such a case, the height of the principal ray having a high angle of view on the cemented surface is higher than in a case where the cemented lens CE is disposed to be closer to the object side in the rear group GR. Thus, it becomes easy to satisfactorily obtain an achromatizing effect. It is preferable that the cemented lens CE consists of two lenses of a positive lens and a negative lens for reduction in size.

As an example, the rear group GR shown in FIG. 1 consists of, in order from the object side to the image side, a third lens L3 of a plano-convex lens of which the surface on the image side is convex, a fourth lens L4 of a biconvex lens, and a fifth lens L5 of a plano-concave lens of which the surface on the object side is concave, and the fourth lens L4 and the fifth lens L5 are cemented to each other to compose the cemented lens CE.

It is preferable that the number of lenses in the objective optical system for an endoscope according to the present disclosure is five. By reducing the number of lenses constituting the objective optical system for an endoscope in this manner, there is an advantage in shortening the total length.

More specifically, it is preferable that the number of lenses in the front group GF is two and the number of lenses in the rear group GR is three. In an optical system having a wide angle of view, by the front group GF comprising two lenses, it is possible to gradually bend rays having a high angle of view as compared with a case where the front group GF comprises only one lens. Thus, the amount of aberration generation can be suppressed. In the rear group GR, as described above, disposing a positive lens to be closest to the object side is advantageous in suppressing spherical aberration, and disposing the cemented lens CE to be closest to the image side is advantageous in suppressing lateral chromatic aberration. That is, in the rear group GR, it is preferable that the positive lens for suppressing spherical aberration is disposed near the aperture stop St, and the cemented lens CE for suppressing lateral chromatic aberration is disposed at a position far from the aperture stop St, separately from the positive lens. From the above, it is preferable that the rear group GR consists of at least three lenses. On the other hand, in order to shorten the total length, it is preferable that the number of lenses is as small as possible. As a result, it is preferable that the number of lenses in the rear group GR is three.

Next, a configuration relating to conditional expressions will be described for each conditional expression. The objective optical system for an endoscope according to the present disclosure satisfies Conditional Expression (1) in a case where a focal length of a whole system is denoted by f and a focal length of the front group GF is denoted by fF. By not allowing a corresponding value of Conditional Expression (1) to be equal to or less than the lower limit, there is an advantage in suppressing field curvature. By not allowing a corresponding value of Conditional Expression (1) to be equal to or greater than the upper limit, there is an advantage in ensuring a back focal length. In a case of a configuration in which Conditional Expression (1-1) is satisfied, it is possible to obtain more favorable characteristics.

−2<f/fF<−1.3 (1)

−1.8<f/fF<−1.1 (1-1)

Further, the objective optical system for an endoscope of the present disclosure satisfies Conditional Expression (2) in a case where a distance on an optical axis from a lens surface closest to the object side to a lens surface closest to the image side is denoted by L and a focal length of a whole system is denoted by f. By not allowing a corresponding value of Conditional Expression (2) to be equal to or less than the lower limit, there is an advantage in ensuring a wide angle of view. By not allowing a corresponding value of Conditional Expression (2) to be equal to or greater than the upper limit, there is an advantage in shortening a total length. In a case of a configuration in which Conditional Expression (2-1) is satisfied, it is possible to obtain more favorable characteristics.

3<L/f<5 (2)

3.2<L/f<4.8 (2-1)

Moreover, it is preferable that the objective optical system for an endoscope according to the present disclosure satisfies Conditional Expression (3) in a case where a focal length of a whole system is denoted by f and a focal length of the first lens L1 is denoted by f1. By not allowing a corresponding value of Conditional Expression (3) to be equal to or less than the lower limit, there is an advantage in suppressing field curvature. By not allowing the corresponding value of Conditional Expression (3) to be equal to or greater than the upper limit, there is an advantage in ensuring a wide angle of view while suppressing an increase in lens diameter. In a case of a configuration in which Conditional Expression (3-1) is satisfied, it is possible to obtain more favorable characteristics.

−0.85<f/f1<−0.3 (3)

−0.8<f/f1<−0.35 (3-1)

In a configuration in which the cemented lens CE included in the rear group GR consists of two lenses, it is preferable that the objective optical system for an endoscope according to the present disclosure satisfies Conditional Expression (4) in a case where an Abbe number of the positive lens constituting the cemented lens CE at a d line is denoted by vp and an Abbe number of the negative lens constituting the cemented lens CE at a d line is denoted by vn. By satisfying Conditional Expression (4), it becomes easy to suppress lateral chromatic aberration. In a case of a configuration in which Conditional Expression (4-1) is satisfied, it is possible to obtain more favorable characteristics.

8<vp−vn<28 (4)

10<vp−vn<26 (4-1)

It is preferable that the objective optical system for an endoscope according to the present disclosure satisfies Conditional Expression (5) in a case where a focal length of a whole system is denoted by f and a focal length of the second lens L2 is denoted by f2. By satisfying Conditional Expression (5), there is an advantage in suppressing field curvature. In a case of a configuration in which Conditional Expression (5-1) is satisfied, it is possible to obtain more favorable characteristics.

−1.2<f/f2<−0.4 (5)

−1.1<f/f2<−0.5 (5-1)

It is preferable that the objective optical system for an endoscope according to the present disclosure satisfies Conditional Expression (6) in a case where a focal length of a whole system is denoted by f and a focal length of the rear group GR is denoted by fR. By not allowing a corresponding value of Conditional Expression (6) to be equal to or less than the lower limit, there is an advantage in suppressing an incidence angle of the principal ray of the off-axis ray on the image plane Sim. By not allowing a corresponding value of Conditional Expression (6) to be equal to or greater than the upper limit, there is an advantage in suppressing distortion. In a case of a configuration in which Conditional Expression (6-1) is satisfied, it is possible to obtain more favorable characteristics.

0.7<f/fR<1.5 (6)

0.8<f/fR<1.4 (6-1)

It is preferable that the objective optical system for an endoscope according to the present disclosure satisfies Conditional Expression (7) in a case where a focal length of a whole system is denoted by f and a focal length of the cemented lens CE of the rear group GR is denoted by fc. By not allowing a corresponding value of Conditional Expression (7) to be equal to or less than the lower limit, there is an advantage in suppressing an incidence angle of the principal ray of the off-axis ray on the image plane Sim. By not allowing a corresponding value of Conditional Expression (7) to be equal to or greater than the upper limit, there is an advantage in suppressing distortion. In a case of a configuration in which Conditional Expression (7-1) is satisfied, it is possible to obtain more favorable characteristics.

0.05<f/fc<0.5 (7)

0.07<f/fc<0.45 (7-1)

The above-mentioned preferred configurations and available configurations may be optional combinations, and it is preferable to selectively adopt the configurations in accordance with required specifications. According to the present disclosure, it is possible to realize an objective optical system for an endoscope which has a wide angle of view, has a small size, and maintains favorable optical performance. Here, the term “a wide angle of view” means that the maximum total angle of view is 100° or more.

Next, examples of the objective optical system for an endoscope according to the present disclosure will be described. In consideration of a state in which an endoscope is used, cross-sectional views, basic lens data, and aberration diagrams relating to all the examples to be described below are obtained in a case where an object at a finite distance is observed. More specifically, an object at a finite distance relating to data pieces of the following examples is an object of which a distance from the object to an object side surface of the parallel flat plate P1 of the front group GF is 4 mm (millimeters) and a radius of curvature of a concave surface on the image side is 4 mm (millimeters).

### EXAMPLE 1

FIG. 1 shows a cross-sectional view illustrating a configuration and rays of an objective optical system for an endoscope of Example 1, and an illustration method thereof is the same as described above. Therefore, repeated description thereof will be partially omitted herein. The objective optical system for an endoscope of Example 1 consists of, in order from the object side to the image side, a front group GF having a negative refractive power, an aperture stop St, and a rear group GR having a positive refractive power. The front group GF consists of, in order from the object side to the image side, a parallel flat plate P1, a first lens L1, and a second lens L2. The rear group GR consists of, in order from the object side to the image side, a third lens L3, a fourth lens L4, and a fifth lens L5. The first lens L1, the second lens L2, and the fifth lens L5 are negative lenses. The third lens L3 and the fourth lens L4 are positive lenses. The first lens L1, the second lens L2, and the third lens L3 are single lenses. The fourth lens L4 and the fifth lens L5 are cemented to each other to compose a cemented lens CE. The above description is the outline of the objective optical system for an endoscope of Example 1.

Table 1 shows basic lens data of the objective optical system for an endoscope of Example 1, and Table 2 shows a specification thereof. In Table 1, the column of Sn shows surface numbers which are obtained in a case where a surface closest to the object side is set as a first surface and a number is increased toward the image side one by one. The column of R shows radii of curvature of the respective surfaces. The column of D shows surface distances on the optical axis between the respective surfaces and surfaces adjacent to the image side. The column of Nd shows a refractive index of each constituent element at a d line, and the column of vd shows an Abbe number of each constituent element at a d line.

In Table 1, the sign of the radius of curvature of the surface convex toward the object side is positive and the sign of the radius of curvature of the surface convex toward the image side is negative. Table 1 also shows the parallel flat plate P1, the aperture stop St, the optical member 4, and the optical member 5 together. In Table 1, in a place of a surface number of a surface corresponding to the aperture stop St, the surface number and a term of (St) are noted. A value at the bottom place of D in Table 1 indicates a distance between the image plane Sim and the surface closest to the image side in the table.

In the range of Table 2, values of the focal length f, the back focal length Bf at an air conversion distance, and the F number FNo., and the maximum total angle of view 2ω are based on a d line. Bf indicates an air conversion distance from a lens surface closest to the image side to an image side focal position. (°) in the place of 2ω indicates that the unit thereof is a degree. Tables 1 and 2 show numerical values rounded off to predetermined decimal places.

TABLE 1
Example 1

| Sn | R | D | Nd | νd |
|---|---|---|---|---|
| 1 | ∞ | 0.2000 | 1.88299 | 40.78 |
| 2 | ∞ | 0.0000 | | |
| 3 | ∞ | 0.1809 | 1.60342 | 38.03 |
| 4 | 0.2730 | 0.0700 | | |
| 5 | −0.2730 | 0.1809 | 1.60342 | 38.03 |
| 6 | ∞ | 0.0350 | | |
| 7(St) | ∞ | 0.0000 | | |
| 8 | ∞ | 0.4000 | 1.88300 | 40.76 |
| 9 | −0.4331 | 0.0200 | | |
| 10 | 0.8791 | 0.3250 | 1.80000 | 29.84 |
| 11 | −0.5000 | 0.1500 | 1.98613 | 16.48 |
| 12 | ∞ | 0.0350 | | |
| 13 | ∞ | 0.3000 | 1.51633 | 64.14 |
| 14 | ∞ | 0.0500 | | |
| 15 | ∞ | 0.4000 | 1.51633 | 64.14 |
| 16 | ∞ | 0.1675 | | |

TABLE 2
Example 1

| | |
|---|---|
| f | 0.35 |
| Bf | 0.69 |
| FNo. | 4.36 |
| 2ω(°) | 107.6 |

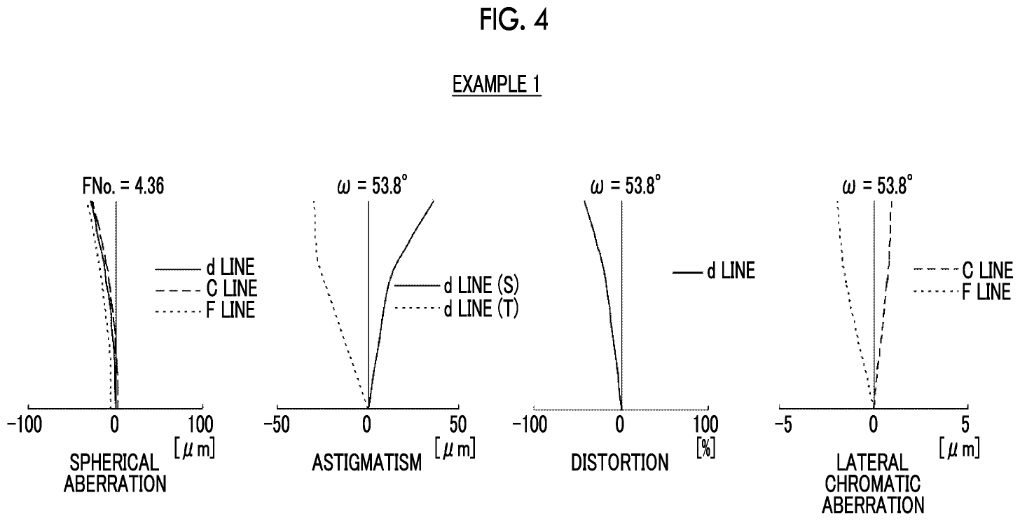

FIG. 4 shows a diagram of aberrations of the objective optical system for an endoscope of Example 1. In FIG. 4, in order from the left side, a spherical aberration diagram, an astigmatism diagram, a distortion diagram, and a lateral chromatic aberration diagram are shown. In the spherical aberration diagram, aberrations at a d line, a C line, and an F line are indicated by a solid line, a long dashed line, and a short dashed line, respectively. In the astigmatism diagram, aberration in a sagittal direction at a d line is indicated by a solid line, and aberration in a tangential direction at a d line is indicated by a short dashed line. In the distortion diagram, aberration at a d line is indicated by a solid line. In the lateral chromatic aberration diagram, aberrations at a C line and an F line are respectively indicated by a long dashed line and a short dashed line. In the spherical aberration diagram, FNo. indicates an F number. In the other aberration diagrams, ω indicates a half angle of view.

Symbols, meanings, description methods, and illustration methods of the respective data pieces relating to Example 1 are the same as those in the following examples unless otherwise noted. Therefore, in the following description, repeated description will be omitted.

### EXAMPLE 2

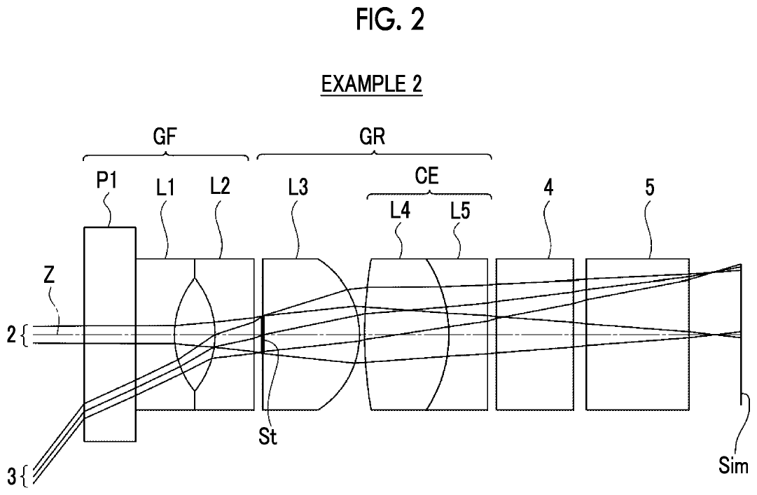

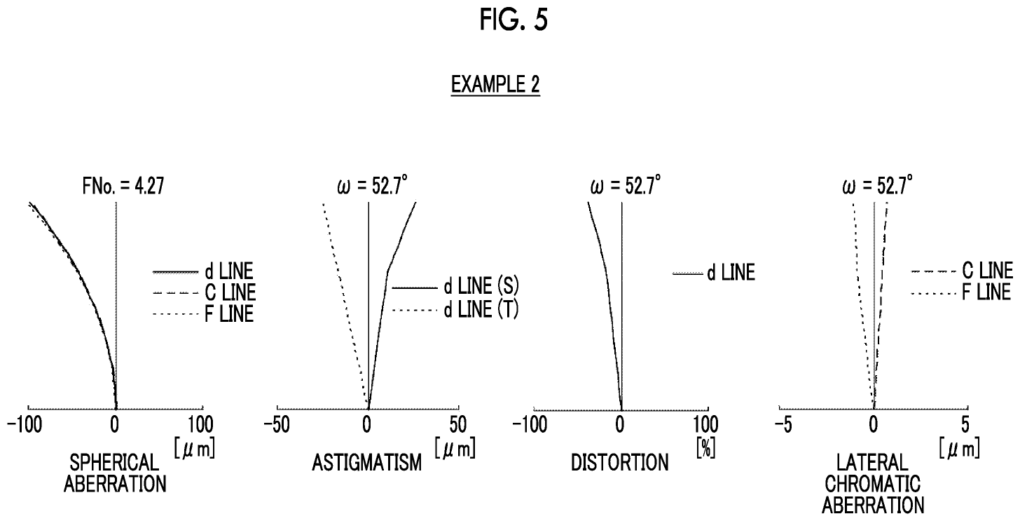

FIG. 2 shows a cross-sectional view illustrating a configuration and rays of an objective optical system for an endoscope of Example 2. The objective optical system for an endoscope of Example 2 has the same configuration as the outline of the objective optical system for an endoscope of Example 1. Table 3 shows basic lens data of the objective optical system for an endoscope of Example 2, Table 4 shows a specification thereof, and FIG. 5 shows a diagram of aberrations.

TABLE 3
Example 2

| Sn | R | D | Nd | νd |
|---|---|---|---|---|
| 1 | ∞ | 0.2000 | 1.88299 | 40.78 |
| 2 | ∞ | 0.0000 | | |
| 3 | ∞ | 0.1512 | 1.80400 | 46.53 |
| 4 | 0.3600 | 0.1580 | | |
| 5 | −0.3600 | 0.1512 | 1.80400 | 46.53 |
| 6 | ∞ | 0.0350 | | |
| 7(St) | ∞ | 0.0000 | | |
| 8 | ∞ | 0.3763 | 1.88300 | 40.76 |
| 9 | −0.3600 | 0.0200 | | |
| 10 | 1.7573 | 0.3258 | 1.83400 | 37.34 |
| 11 | −0.5547 | 0.1500 | 1.98613 | 16.48 |
| 12 | ∞ | 0.0350 | | |
| 13 | ∞ | 0.3000 | 1.51633 | 64.14 |
| 14 | ∞ | 0.0500 | | |
| 15 | ∞ | 0.4000 | 1.51633 | 64.14 |
| 16 | ∞ | 0.2023 | | |

TABLE 4
Example 2

| | |
|---|---|
| f | 0.34 |
| Bf | 0.72 |
| FNo. | 4.27 |
| 2ω(°) | 105.4 |

### EXAMPLE 3

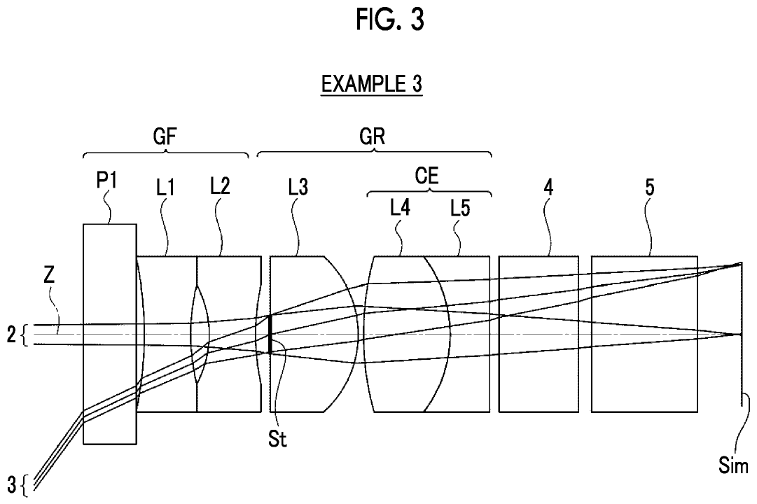

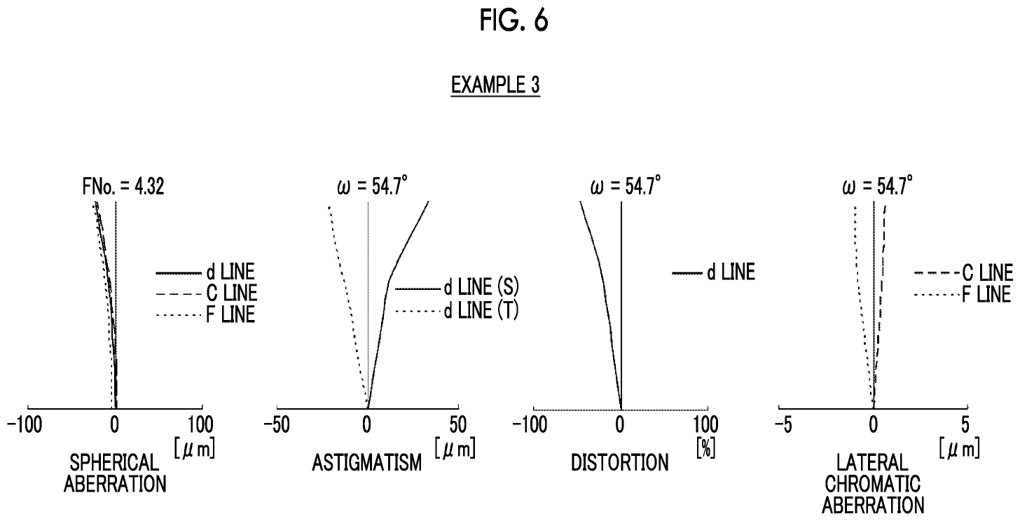

FIG. 3 shows a cross-sectional view illustrating a configuration and rays of an objective optical system for an endoscope of Example 3. The objective optical system for an endoscope of Example 3 has the same configuration as the outline of the objective optical system for an endoscope of Example 1. Table 5 shows basic lens data of the objective optical system for an endoscope of Example 3, Table 6 shows a specification thereof, and FIG. 6 shows a diagram of aberrations.

TABLE 5
Example 3

| Sn | R | D | Nd | νd |
|---|---|---|---|---|
| 1 | ∞ | 0.2000 | 1.88299 | 40.78 |
| 2 | ∞ | 0.0300 | | |
| 3 | −1.4800 | 0.1756 | 1.67790 | 55.34 |
| 4 | 0.8012 | 0.0700 | | |
| 5 | −0.4114 | 0.1756 | 1.72916 | 54.68 |
| 6 | 0.9155 | 0.0550 | | |
| 7(St) | ∞ | 0.0000 | | |
| 8 | ∞ | 0.3327 | 1.85150 | 40.78 |
| 9 | −0.4093 | 0.0200 | | |
| 10 | 1.1541 | 0.3250 | 1.95375 | 32.32 |
| 11 | −0.5000 | 0.1500 | 1.98613 | 16.48 |
| 12 | ∞ | 0.0350 | | |
| 13 | ∞ | 0.3000 | 1.51633 | 64.14 |
| 14 | ∞ | 0.0500 | | |
| 15 | ∞ | 0.4000 | 1.51633 | 64.14 |
| 16 | ∞ | 0.1670 | | |

TABLE 6
Example 3

| | |
|---|---|
| f | 0.37 |
| Bf | 0.68 |
| FNo. | 4.32 |
| 2ω(°) | 109.4 |

Table 7 shows values of the objective optical systems for an endoscope of Examples 1 to 3 corresponding to Conditional Expressions (1) to (7). In Examples 1 to 3, a d line is used as a reference wavelength. Table 7 shows the values on a d line basis.

TABLE 7

| Expression | | | | |
|---|---|---|---|---|
| number | | Example 1 | Example 2 | Example 3 |
| (1) | f/fF | −1.660 | −1.786 | −1.672 |
| (2) | L/f | 3.906 | 4.023 | 3.548 |
| (3) | f/f1 | −0.771 | −0.759 | −0.494 |
| (4) | νp − νn | 13.36 | 20.86 | 15.84 |
| (5) | f/f2 | −0.771 | −0.759 | −0.997 |
| (6) | f/fR | 0.959 | 0.946 | 1.044 |
| (7) | f/fc | 0.209 | 0.076 | 0.283 |

As can be seen from the above-mentioned data, in the objective optical systems for an endoscope of Examples 1 to 3, the number of lenses is five, the total length and the outer diameter are reduced, or the maximum total angle of view is a wide angle of 105° or more. Further, in the objective optical systems for an endoscope of Examples 1 to 3, the F number is smaller than 4.5, and high optical performance is achieved by satisfactorily correcting various aberrations.

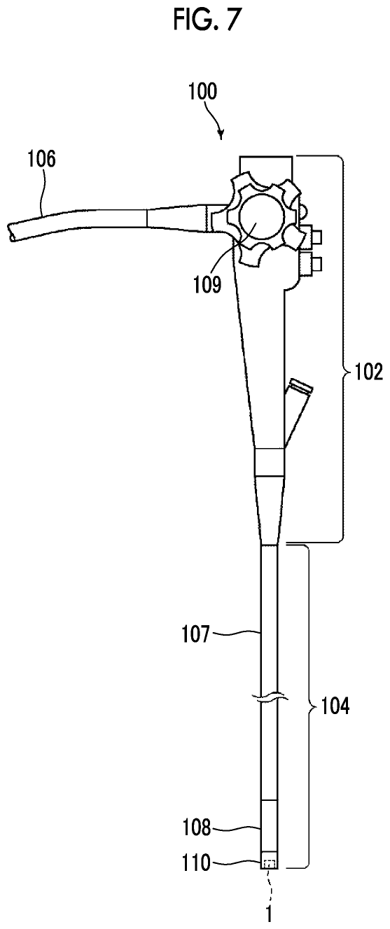

Next, an endoscope according to an embodiment of the present disclosure will be described. FIG. 7 shows a schematic overall configuration diagram of an endoscope according to an embodiment of the present disclosure. An endoscope 100 shown in FIG. 7 mainly comprises an operation unit 102, an insertion part 104, and a universal cord 106 that is to be connected to a connector part (not shown). A large portion of the insertion part 104 is a soft portion 107 that is bendable in any direction along an insertion path, a bendable portion 108 is connected to the distal end of the soft portion 107, and a distal end portion 110 is connected to the distal end of the bendable portion 108. The bendable portion 108 is provided to allow the distal end portion 110 to turn in a desired direction, and can be operated to be bent by the rotational movement of a bending operation knob 109 provided on the operation unit 102. An objective optical system 1 for an endoscope according to the embodiment of the present disclosure is provided in the distal end of the distal end portion 110. FIG. 7 schematically shows the objective optical system 1 for an endoscope. An imaging element (not shown) such as a charge coupled device (CCD) or a complementary metal oxide semiconductor (CMOS) that outputs an imaging signal is disposed on an image plane of the objective optical system 1 for an endoscope. Since the endoscope according to the present disclosure comprises the objective optical system for an endoscope according to the embodiment of the present disclosure, the diameter of the insertion part 104 can be reduced, the endoscope can make an observation with a wide angle of view, and a favorable image can be obtained.

The technology of the present disclosure has been hitherto described through the embodiments and the examples, but the technology of the present disclosure is not limited to the above-mentioned embodiments and examples, and may be modified into various forms. For example, values such as the radius of curvature, the surface distance, the refractive index, and the Abbe number of each lens are not limited to the values shown in the examples, and different values may be used therefor.

What is claimed is:

1. An objective optical system for an endoscope consisting of, in order from an object side to an image side: a front group having a negative refractive power; an aperture stop; and a rear group having a positive refractive power,
wherein a lens closest to the object side in the front group is a first lens having a negative refractive power and concave toward the image side,
a lens positioned second from the object side in the front group is a second lens having a negative refractive power and concave toward the object side,
the rear group includes a cemented lens in which a positive lens and a negative lens are cemented in order from the object side, and
Conditional Expressions (1) and (2) are satisfied in a case where a focal length of the objective optical system for an endoscope is denoted by f, a focal length of the front group is denoted by fF, and a distance on an optical axis from a lens surface closest to the object side to a lens surface closest to the image side is denoted by L,
−2<f/fF<−1.3 (1)
3<L/f<5 (2).

2. The objective optical system for an endoscope according to claim 1, wherein Conditional Expression (3) is satisfied in a case where a focal length of the first lens is denoted by f1,
−0.8<f/f1<−0.3 (3).

3. The objective optical system for an endoscope according to claim 1,
wherein the cemented lens consists of two lenses, and
Conditional Expression (4) is satisfied in a case where an Abbe number of the positive lens constituting the cemented lens at a d line is denoted by vp and an Abbe number of the negative lens constituting the cemented lens at a d line is denoted by vn,
8<vp−vn<28 (4).

4. The objective optical system for an endoscope according to claim 1,
wherein Conditional Expression (5) is satisfied in a case where a focal length of the second lens is denoted by f2,
−1.2<f/f2<−0.4 (5).

5. The objective optical system for an endoscope according to claim 1,
wherein Conditional Expression (6) is satisfied in a case where a focal length of the rear group is denoted by fR,
0.7<f/fR<1.5 (6).

6. The objective optical system for an endoscope according to claim 1,
wherein Conditional Expression (7) is satisfied in a case where a focal length of the cemented lens is denoted by fc,
0.5<f/fc<0.5 (7).

7. The objective optical system for an endoscope according to claim 1,
wherein the cemented lens is disposed to be closest to the image side in the rear group.

8. The objective optical system for an endoscope according to claim 1,
wherein the second lens is a plano-concave lens or a biconcave lens.

9. The objective optical system for an endoscope according to claim 1,
wherein a lens closest to the object side in the rear group is a positive lens.

10. The objective optical system for an endoscope according to claim 1,
wherein the front group includes a parallel flat plate closest to the object side.

11. The objective optical system for an endoscope according to claim 1,
wherein the number of lenses in the objective optical system for an endoscope is five.

12. The objective optical system for an endoscope according to claim 1,
wherein the number of lenses in the front group is two, and
the number of lenses in the rear group is three.

13. The objective optical system for an endoscope according to claim 1,
wherein Conditional Expression (1-1) is satisfied,
−1.8<f/fF<−1.1 (1-1).

14. The objective optical system for an endoscope according to claim 1,
wherein Conditional Expression (2-1) is satisfied,
3.2<L/f<4.8 (2-1).

15. The objective optical system for an endoscope according to claim 2,
wherein Conditional Expression (3-1) is satisfied,
−0.8<f/f1<−0.35 (3-1).

16. The objective optical system for an endoscope according to claim 3,
wherein Conditional Expression (4-1) is satisfied,
10<vp−vn<26 (4-1).

17. The objective optical system for an endoscope according to claim 4,
wherein Conditional Expression (5-1) is satisfied,
−1.1<f/f2<−0.5 (5-1).

18. The objective optical system for an endoscope according to claim 5,
wherein Conditional Expression (6-1) is satisfied,
0.8<f/fR<1.4 (6-1).

19. The objective optical system for an endoscope according to claim 6,
wherein Conditional Expression (7-1) is satisfied,
0.07<f/fc<0.45 (7-1).

20. An endoscope comprising:
the objective optical system for an endoscope according to claim 1.
